(19) United States
(12) Patent Application Publication
(10) Pub. No.: US 2004/0266809 A1
(43) Pub. Date: Dec. 30, 2004

(54) Method of treating multiple myeloma

(76) Inventors: David Emanuel, Tenafly, NJ (US); Sumant Ramachandra, East Brunswick, NJ (US)

Correspondence Address: Patrick G. Gattari, McDonnell Boehnen Hulbert & Berghoff LLP, 32nd Floor, 300 S. Wacker Drive, Chicago, IL, 60606, US

(21) Appl. No.: 10/847,847

(22) Filed: May 18, 2004

Related U.S. Application Data
(60) Provisional application No. 60/471,601, filed on May 19, 2003.

(51) Int. Cl. A61K 31/4745

(52) U.S. Cl. 514/283

(57) ABSTRACT

Multiple myeloma can be treated by administration of irinotecan combined with revimid. The present invention includes methods of treating multiple myeloma by administering the combination of irinotecan and revimid, as well as pharmaceutical kits.

### CROSS-REFERENCE TO RELATED APPLICATION

This application claims priority to U.S. Provisional Patent Application Ser. No. 60/471,601, filed May 19, 2003.

### BACKGROUND OF THE INVENTION

1. Field of the Invention

The present invention relates to a method for treating a cancer, especially multiple myeloma, in a subject in need of such a treatment, said method comprising administering to the patient a therapeutically effective amount of irinotecan and revimid. The invention also relates to compositions or packaged units comprising irinotecan and revimid.

2. Description of Related Art

Multiple myeloma is a disseminated malignancy of plasma cells that affects approximately 14,600 new patients each year in the United States. The etiology of this rare blood disease, affecting mainly the middle-aged to elderly population, is largely unknown although genetic predisposition and environmental factors have been implicated. From onset, malignant plasma cells arising from clonal expansion accumulate in the bone marrow, producing abnormally high levels of immunoglobulins. Multiple myeloma is difficult to diagnose early because there may be no symptoms in early stage. Bone pain especially secondary to compression fractures of the ribs or vertebrae is the most common symptom.

Even if the combination of melphalan and prednisone (MP) and variations of the combination of vincristine, doxorubicin, and dexamethasone (VAD) are the 2 most commonly used regimens for first-line treatment of the disease, no effective long-term treatment exists for multiple myeloma.

In selected patients, usually <70 years of age, high-dose chemotherapy supported by autologous stem cell transplantation (ASCT) may prolong event-free survival if the procedure is performed within 12 months of initial diagnosis. However almost all patients receiving high-dose chemotherapy and an autologous peripheral stem cell transplant will ultimately relapse.

Newer therapeutic strategies for multiple myeloma, are clearly needed.

It has now been found, and this forms the subject of the present invention, that patients with multiple myeloma, especially patients who have failed prior chemotherapy, could realize significant clinical benefit by having access to a combined treatment with irinotecan and revimid.

Among the several advantages found to be achieved by the present invention, therefore, may be noted the provision of an effective method for the treatment of multiple myeloma, the provision of such methods that provided beneficial properties that are comparable to or superior to those provided by known and conventional methods of treatment for these conditions, and the provision of compositions, pharmaceutical compositions and kits to effect these methods.

It is therefore a first object of the present invention combined preparations for treating multiple myeloma, comprising irinotecan administered in combination with revimid.

### SUMMARY OF THE INVENTION

In one aspect, the invention is directed to method for treating multiple myeloma in a animal subject by administering irinotecan in combination with revimid. In another aspect, the patient has had prior chemotherapy and, in a further aspect, the patient demonstrated failure of the prior chemotherapy. The irinotecan and revimid may be administered sequentially, in any order, or simultaneously. The invention is also directed to pharmaceutical preparations including irinotecan and revimid

### DETAILED DESCRIPTION

Irinotecan [1,4′-Bipiperidine]-1′-carboxylic acid (4S)-4,11-diethyl-3,4,12,14-tetrahydro-4-hydroxy-3,14-dioxo-1H-pyrano[3′,4′:6,7]indolizino[1,2-b]quinolin-9-yl ester (CAS RN 97682-44-5) is a camptothecin analog and topoisomerase-I inhibitor derived from a compound, which occurs naturally in the Chinese tree, Camptotheca acuminata. Irinotecan can be prepared following the procedure disclosed in U.S. Pat. No. 4,604,463, European patent No. 835,257 or S. Sawada et al., Chem. Pharm. Bull. 39, 1446 (1991). Irinotecan hydrochloride, clinically investigated as CPT-11, is a commercially available compound (CAMPTOSAR™, Pharmacia Corp.).

As used herein the term irinotecan encompasses all pharmaceutically acceptable salts of irinotecan, particularly the hydrochloride salt.

In a preferred aspect of the present invention, irinotecan is irinotecan hydrochloride, namely CPT-11.

Revimid, also known as CDC-501 or CC-5013, is the lead compound in a series of thalidomide derivatives that inhibit TNF-alpha overproduction, developed by Celgene for the potential treatment of hematological and solid tumor cancers and inflammatory diseases. Revimid can be prepared following the procedure reported in U.S. Pat. No. 5,635,517.

Approximately 50% of patients with newly diagnosed myeloma have disease that is unresponsive to either with melphalan and prednisone (MP) or vincristine, doxorubicin and dexamethasone (VAD). Even in patients who initially respond to treatment and receive further consolidation therapy almost all will ultimately relapse. For patients who relapse following induction therapy with MP, VAD is the second-line treatment of choice. In patients with VAD-refractory disease, high-dose alkylating agents either used alone or in combination, e.g. cyclophesphamide plus etoposide or combinations of etoposide, dexamethasone, doxorubicin and platinum can induce a response in approximately one-third of patients, albeit for short duration.

Combined preparations according to the invention are particularly effective in case of relapsed or refractory multiple myeloma.

It is therefore a further object of the present invention combined preparations for treating multiple myeloma in a patient who demonstrated failure of prior treatment with prior chemotherapy, which comprises administering a therapeutically effective amount of irinotecan.

In the present specification “prior chemotherapy” means a treatment of newly diagnosed previously untreated patients with MP, VAD or an alkylating agent either used alone or in combination, e.g. cyclophosphamide plus etoposide or combinations of etoposide, dexamethasone, doxorubicin.

The present invention particularly relates to combined preparations for treating multiple myeloma in a patient who demonstrated failure of prior treatment with combinations of MP, VAD, cyclophosphamide and etoposide or etoposide, dexamethasone and doxorubicin.

The administration of the constituents of the combined preparations of the present invention can be made simultaneously, separately or sequentially in any order. Namely, the present invention intends to embrace administration of irinotecan and revimid in a sequential manner in a regimen that will provide beneficial effects of the drug combination, and intends as well to embrace co-administration of these agents in a substantially simultaneous manner, such as in a single dosage device having a fixed ratio of these active agents or in multiple, separate dosage devices for each agent, where the separate dosage devices can be taken together contemporaneously, or taken within a period of time sufficient to receive a beneficial effect from both of the constituent agents of the combination.

It is therefore another object of the present invention the use of irinotecan in combination with revimid for the preparation of a medicament for simultaneous, separate or sequential use for the treatment of multiple myeloma in a patient who demonstrated failure of prior chemotherapy, particularly in a patient who demonstrated failure of prior treatment with combinations of MP, VAD, cyclophosphamide and etoposide or etoposide, dexamethasone and doxorubicin.

The constituents of the combined preparations according to the invention can be administered to a patient in any acceptable manner that is medically acceptable including orally, parenterally, or with locoregional therapeutic approaches such as, e.g., implants. Oral administration includes administering the constituents of the combined preparation in a suitable oral form such as, e.g., tablets, capsules, lozenges, suspensions, solutions, emulsions, powders, syrups and the like. Parenteral administration includes administering the constituents of the combined preparation by subcutaneous, intravenous or intramuscular injections. Implants include intra-arterial implants, for example an intra-hepatic artery implant.

Preferably, irinotecan may be administered orally in the form of a pharmaceutically acceptable formulation for oral administration, which can provide a means for protracted drug exposure to actively cycling malignant cells with greater convenience and potentially lower costs. In general, the pharmaceutically acceptable formulations for oral administration according to the present invention may comprise a therapeutically effective amount of irinotecan in combination with a pharmaceutically acceptable carrier or diluent. Examples of oral formulations include solid oral preparations such as, e.g., tablets, capsules, powders and granules, and liquid oral preparations such as e.g., solutions and suspensions, that may be prepared following conventional literature or common techniques well known to those skilled in the art.

Suitable oral dosage forms according to the present invention may be prepared, for example, as described in the Pharmacia & Upjohn S.p.A. International patent application WO 01/10443 filed on Jul. 11, 2000, Teva Pharm. Ind. LTD U.S. patent application Ser. No. 20020147208 filed on Dec. 20, 2001 and Pharmacia Italia S.p.A. International patent application WO 01/30351 filed on Oct. 2, 2000.

Preferably, revimid may be administered orally.

A further aspect of the present invention is to provide a method for the treatment of multiple myeloma in a patient in need of such a treatment, the method comprising administering to said patient a therapeutically effective amount of irinotecan and revimid.

In a particular aspect, the patient is a patient who demonstrated failure of prior chemotherapy, especially a patient who demonstrated failure of prior treatment with combinations of MP, VAD, cyclophosphamide and etoposide or etoposide, dexamethasone and doxorubicin.

In the method of the subject invention, irinotecan may be administered simultaneously with revimid, or the compounds may be administered sequentially, in either order. It will be appreciated that the actual preferred method and order of administration will vary according to, inter alia, the particular formulation of irinotecan being utilized, the particular formulation of revimid being utilized, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. A therapeutically effective amount of a pharmaceutical agent is that which provides an objectively identifiable improvement as noted by the clinician or other qualified observer. Regression of a tumor in a patient is typically measured with reference to the diameter of a tumor. Decrease in the diameter of a tumor indicates regression. Regression is also indicated by failure of tumors to reoccur after treatment has stopped.

In the method according to the present invention, the amount of irinotecan, together with the amount of revimid, constitute an amount therapeutically effective for the treatment of multiple myeloma.

The dosage regimen should be preferably tailored to the patient's conditions and response and may need to be adjusted in response to changes in conditions.

In the present specification the term “failure of treatment” includes progression of disease while receiving a chemotherapy regimen without experiencing any transient improvement, no objective response after receiving one or more cycles of a chemotherapy regimen and a limited response with subsequent progression, while receiving a chemotherapy regimen.

In the present specification “therapeutically effective amount” means, unless otherwise indicated, the amount of drug that is required to be administered to achieve the desired therapeutic effect.

In the present specification “refractory cancer” means, unless otherwise specified, a cancer that has not responded to treatment.

In the present specification “regimen” means, unless otherwise specified, a treatment plan that specifies the dosage, the schedule, and the duration of treatment.

In the present specification “relapse” means, unless otherwise specified, the return of signs and symptoms of cancer after a period of improvement.

What is claimed is:

1. A method for treating multiple myeloma in a subject comprising administering irinotecan in combination with revimid.

2. The method of claim 1, wherein the irinotecan is a pharmaceutically acceptable salt.

3. The method of claim 2, wherein the pharmaceutically acceptable salt of irinotecan is a hydrochloride salt.

4. The method of claim 3, wherein the irinotecan hydrochloride salt is CPT-11.

5. The method of claim 1, wherein the irinotecan is administered as an oral dosage form.

6. The method of claim 1, wherein the revimid is administered as an oral dosage form.

7. The method of claim 1, wherein the irinotecan and revimid are administered sequentially.

8. The method of claim 7, where the irinotecan and revimid are administered sequentially in any order.

9. The method of claim 1, wherein the irinotecan and revimid are administered simultaneously.

10. The method of claim 1, wherein the subject has had prior chemotherapy.

11. The method of claim 10 wherein the prior chemotherapy comprises treatment with MP, treatment with VAD, or treatment with one or more alkylating agents.

12. The method of claim 10 wherein the prior chemotherapy comprises treatment with cyclophosphamide and etoposide, or treatment with etoposide, dexamethasone and doxorubicin.

13. The method of claim 10 wherein irinotecan and revimid are administered sequentially, in any order.

14. The method of claim 10 wherein irinotecan and revimid are administered simultaneously.

15. A pharmaceutical preparation for the treatment of multiple myeloma comprising irinotecan and revimid.

16. The pharmaceutical preparation of claim 15 wherein the irinotecan and the revimid are provided in a single dosage device.

17. The pharmaceutical preparation of claim 15 wherein the irinotecan and the revimid are provided in separate dosage devices.

18. A method for treating multiple myeloma in a patient comprising administering oral irinotecan in combination with oral revimid.

19. The method of claim 18, wherein the irinotecan is a pharmaceutically acceptable salt form.

20. The method of claim 19, wherein the pharmaceutically acceptable salt form of irinotecan is a hydrochloride salt.

21. The method of claim 20, wherein the irinotecan hydrochloride salt is CPT-11.

22. The method of claim 18, wherein the irinotecan and revimid are administered sequentially.

23. The method of claim 22, where the irinotecan and revimid are administered sequentially in any order.

24. The method of claim 18, wherein the irinotecan and revimid are administered simultaneously.
